(19) United States
(12) Patent Application Publication
(10) Pub. No.: US 2005/0075598 A1
(43) Pub. Date: Apr. 7, 2005

(54) Method and apparatus for the measurement of real time drug delivery through the use of a wearable monitor and sensor attached to a transdermal drug delivery device

(76) Inventor: Bruce K. Redding JR., Broomall, PA (US)

Correspondence Address: REED SMITH LLP, 2500 ONE LIBERTY PLACE, 1650 MARKET STREET, PHILADELPHIA, PA, 19103, US

(21) Appl. No.: 10/782,378

(22) Filed: Feb. 18, 2004

Related U.S. Application Data
(63) Continuation-in-part of application No. 09/939,435, filed on Aug. 24, 2001, now abandoned.
(60) Provisional application No. 60/447,920, filed on Feb. 19, 2003.

(51) Int. Cl. A61B 17/20

(52) U.S. Cl. 604/22

(57) ABSTRACT

A transdermal substance delivery device, comprising at least one ultrasonic transducer for generating at least one ultrasonic transmission for inducing movement of at least one substance into a tissue the at least one ultrasonic transmission and the at least one sensor positioned with the at least one transducer to sense reflected ultrasound transmissions, wherein the sensed ultrasound transmissions are indicative of substance actually moved into the tissue. A method for transdermal substance delivery, comprising generating at least one ultrasonic transmission from at least one ultrasonic transducer for inducing movement of at least one substance into a tissue and positioning the at least one ultrasonic transmission and at least one sensor positioned with the at least one transducer to sense reflected ultrasonic transmissions and wherein, the sensed ultrasonic transmissions are indicative of substance actually moved into the tissue.

### CROSS REFERENCE TO RELATED APPLICATIONS

[1] This Application claims priority of copending U.S. Provisional Application Ser. No. 60/447,920, entitled “METHOD AND APPARATUS FOR THE MEASUREMENT OF REAL TIME DRUG DELIVERY THROUGH THE USE OF A WEARABLE MONITOR AND SENSOR ATTACHED TO A TRANSDERMAL DRUG DELIVERY DEVICE”, filed Feb. 19, 2003. This application also is a continuation in part of each of: U.S. patent application Ser. No. 09/939,435, filed Aug. 24, 2001 entitled “ULTRASONICALLY ENHANCED SUBSTANCE DELIVERY METHOD”, U.S. patent application Ser. No. 09/939,506, filed Aug. 24, 2001 entitled “SUBSTANCE DELIVERY SYSTEM”, U.S. patent application Ser. No. 09/939,507, filed Aug. 24, 2001 entitled “ULTRASONICALLY ENHANCED SUBSTANCE DELIVERY SYSTEM AND DEVICE”, and U.S. patent application Ser. No. 10/345,825, filed Jan. 16, 2003 entitled “SUBSTANCE DELIVERY DEVICE”, the entire disclosures of which are each respectively hereby incorporated by reference herein as if being set forth in their respective entireties.

### BACKGROUND OF THE INVENTION

1. Field of the Invention

The present invention relates generally to transdermal substance delivery, and more specifically to a sensor, which can be attached to a wearable transdermal drug delivery device (TDD).

2. Description of the Background

Transdermal delivery systems may employ a medicated device or patch, which may be affixed to an exposed surface of the skin of a patient, thus avoiding the need and the pain associated with drug injections and intravenous drug administration. Transdermal delivery also avoids gastrointestinal metabolism of administered drugs, reducing the elimination of drugs by the liver, and providing a sustained release of the administrated drug. Transdermal delivery may also enhance a patient's compliance with a drug regimen due in part to the relative ease of administration and the sustained release of the medicines.

TDD systems generally rely on pharmaceutical compounds of a molecular weight below 4,000 Daltons. Compounds above 4,000 Daltons in molecular weight may be difficult or impossible to administer transdermally without the aid of electronic, mechanical, or ultrasonic aids.

Passive TDD's have variable delivery rates depending upon a patient's skin structure, placement on the body and the fat content of the patient. Therefore, it is typically difficult to control the dose actually absorbed through the skin of an individual patient. Additionally, current TDD's have no indication of the quantity, or dose, remaining in the patch in a given period of time. There still remains no effective means of gathering dose measurements from TDD's or patch products. Thus, a need exists for a system designed to measure real-time drug delivery through the use of a portable monitor and sensor attached to a transdermal drug delivery device.

### SUMMARY OF THE INVENTION

A transdermal substance delivery device, comprising at least one ultrasonic transducer for generating at least one ultrasonic transmission for inducing movement of at least one substance into a tissue the at least one ultrasonic transmission and the at least one sensor positioned with the at least one transducer to sense reflected ultrasound transmissions, wherein the sensed ultrasound transmissions are indicative of substance actually moved into the tissue. A method for transdermal substance delivery, comprising generating at least one ultrasonic transmission from at least one ultrasonic transducer for inducing movement of at least one substance into a tissue and positioning the at least one ultrasonic transmission and at least one sensor positioned with the at least one transducer to sense reflected ultrasonic transmissions and wherein, the sensed ultrasonic transmissions are indicative of substance actually moved into the tissue.

### BRIEF DESCRIPTION OF THE DRAWINGS

Understanding of the present invention will be facilitated by consideration of the following detailed description of the preferred embodiments of the present invention taken in conjunction with the accompanying drawings, in which numerals refer to like parts, and wherein:

FIG. 1 illustrates an embodiment of the present invention;

FIG. 2A illustrates an embodiment of the present invention;

FIG. 2B illustrates an enlarged view of an embodiment of the present invention;

FIG. 3 illustrates an embodiment of the present invention as worn by a patient; and

FIG. 4. Illustrates the use of an alternating waveform, a conversion from sawtooth to square wave, as generated by the frequency driver of the present invention;

FIG. 5 is an illustration of the structure of human skin;

FIG. 6 illustrates a cross sectional view of an embodiment of a transducer element used with the present invention;

FIG. 7 illustrates an embodiment of a transducer of the present invention, and the use of a polymer potting used as a resonance compatible coupling agent coating over the surface of the transducer element;

FIG. 8. Illustrates an array of transducers that may be used in an embodiment to the present invention to enhance sonic efficiency and to provide multiple sensor sites to the skin;

FIG. 9 is an illustration of multiple transducers which may create an echo sensor return pattern for use with an embodiment of the present invention;

FIG. 10 illustrates a cross sectional view of a modified transdermal patch in accordance with the present invention;

FIG. 11 illustrates an embodiment of a modified transdermal patch in accordance with the present invention;

FIG. 12 is an illustration of an embodiment of the present invention;

FIGS. 13A, 13B, and 13C illustrates an embodiment of a transducer coupler and patch cap of the present invention;

FIG. 14 illustrates an embodiment of a transducer coupler of the present invention;

FIG. 15 illustrates a bottom view of an embodiment of a patch cap of the present invention; and

FIG. 16 illustrates a top view of an embodiment of a patch cap of the present invention.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in a typical drug delivery devices. Those of ordinary skill in the art will recognize that other elements are desirable and/or required in order to implement the present invention. However, because such elements are well known in the art, and because they do not facilitate a better understanding of the present invention, a discussion of such elements is not provided herein. The disclosure hereinbelow is directed to all such variations and modifications to these technologies known, and as will be apparent, to those skilled in the art.

According to an aspect of the present invention, a device for measuring in real-time the effectiveness of transdermal drug delivery by the use of an ultrasound sensor communicatively coupled to a control device may be provided. A portable, programmable and ultrasonic sensor, which may be placed directly in contact with a transdermal delivery device or patch for the purpose of sensing, and controlling the delivery of medications contained within the patch into and through the skin layers of a patient may be provided. The sensor may be placed directly within a drug-containing TDD or may, alternatively, be worn over a transdermal patch, and may be held in place by adhesives, body affixing straps or other suitable materials and/or devices.

A TDD may contain, for example, one or more medications for the treatment of disease or the relief of pain. The sensor, when activated may, by its internal timing circuitry, generate an ultrasonic vibration or sonic transmission through the TDD, causing an echo pattern, which may be received by a transducer receiver. The electronic character of the echo pattern may measure the starting dosage amount within the TDD, and later compare that starting value to later values as the medicant is liberated from the TDD over time. Likewise, the sensor, when activated by internal timing circuitry, may generate an ultrasonic vibration or sonic transmission through the skin of a patient, causing an echo pattern, which may be received by a transducer receiver. The electronic character of this echo pattern may measure the dose which actually permeates the skin, and may later be compared to the electronic character signature starting value within the TDD to the later received values as the medicant is liberated from the TDD over time to calculate the quantity of medicant actually received by the patient at any particular point in time.

According to an aspect of the present invention, a sensor may be attached to a TDD or transdermal patch, which may enable the measurement of real time drug delivery through the TDD or transdermal patch into a patients skin as the active substance within the TDD is deposited or absorbed into the skin of the patient.

According to an aspect of the present invention, a sensor may be attached to a TDD, which may enable the measurement of the quantity of an active substance stored within a TDD, for the purpose of determining the quantity which has been delivered from the TDD and also functioning as a “fuel gauge” for determining the remaining quantity of the active within the TDD, and therefore the remaining “life” of the TDD.

According to an aspect of the present invention, a cymbal type transducer or transducer array (flextensional class V) may be used as an ultrasonic sensor device to deliver either low or high frequency ultrasound transmissions through the TDD for the purpose of functioning as a “fuel gauge” indicator of the drug remaining within the TDD at any point in time.

According to an aspect of the present invention, a cymbal type transducer or transducer array (flextensional class V) may be used in the sensor device to deliver either low or high frequency ultrasound transmissions through the patients skin to measure the amount of the active substance actually delivered through the patients skin structure, in real-time, as the active substance is being delivered by the TDD or absorbed from the TDD by the skin of the patient, for the purpose of providing data to a control device which will determine and record the actual drug quantity, timing and other related factors to a drug delivery regimen for that individual patient.

The present invention may also provide a control device or monitor which may enable: the measurement of the amount of the active substance delivery through the patients skin structure, in real time, as the active substance is being delivered by a TDD or transdermal patch, or absorbed from the TDD by the skin of the patient; measure and record the amount of the active substance remaining within the TDD for the purpose of functioning as a fuel gauge; for measuring and recording the amount of the active substance remaining which was originally stored within the TDD and which has now been deposited or liberated from the TDD for the purpose of functioning as a fuel gauge in a control function; and, determining and recording the actual drug quantity, timing, control and other related factors related to a drug delivery device.

According to an aspect of the present invention, a rechargeable battery may be located in the device or strap, and may be lightweight and thin and capable of providing suitable power to at least one sensor and at least one transducer array.

According to an aspect of the present invention, at least one ultrasonic based dosage sensor may operate by measuring a density change of the skin as it is scanned by an ultrasonic signal emitted from the sensor, by relying upon the application of various ultrasound frequencies, intensities and/or phase modulations to generate a sonic pulse and echo wave, which when received by the sensor may allow for the accurate measurement of a dose delivered through the skin of the patient or the dosage remaining within the TDD. In particular, acoustical energy may be delivered by a portable, self-powered, programmable ultrasonic transducer placed over a medicament containing TDD allowing for a measured dose within the patch to cross the skin barrier, as part of a dose control system.

According to an aspect of the present invention, at least one sensor including a transducer or array of transducers may be built into the TDD or may be connected to the TDD by any appropriate means.

According to an aspect of the present invention, an ultrasonic phase modulation and alternating waveforms and frequency modulation may be used to achieve the transdermal sensor measurement functions as mentioned above.

According to an aspect of the present invention, a combination of ultrasound with ionophoresis, electroporation, depilatories, or with chemical enhancers may be used to facilitate the transdermal sensor measurement functions as mentioned above.

According to an aspect of the present invention, the sensing capability for each drug or substance, which may be delivered transdermally, may be optimized or customized to allow for dose measurement, dose control and to record the dose actually delivered to the patient from a TDD worn on the skin of the patient. The molecular structure of each drug or active substance is different and responds differently to ultrasound. Varying the frequency, intensity, waveform and timing of a sonic or ultrasonic transmission may optimize the measurement of each drug compound stored within a TDD providing crucial information of dose measurement and control including, but not limited to: the quantity of the drug actually absorbed into the skin transdermally, to dose delivered at a particular date, and time; the quantity of the drug liberated from the transdermal drug delivery device or transdermal patch worn on the skin of the patient; and the quantity of the drug remaining within the transdermal drug delivery device or transdermal patch.

According to an aspect of the present invention, use of a dose sensor of this invention may serve to improve the quality of life for patients with diseases or conditions which require periodic administration of drugs by permitting the patients to continually and accurately measure the dose actually delivered to the patient over time. Such sensors can be connected to control devices for the accurate administration of medicinal doses and the recording of the doses actually delivered to the patient. With such dose control and monitoring a more appropriate and customized medication regimen can be established for that individual patient. In addition, the cognitively impaired, elderly, and very young may receive medication with much less supervision, while being absolutely certain of the dose they actually received.

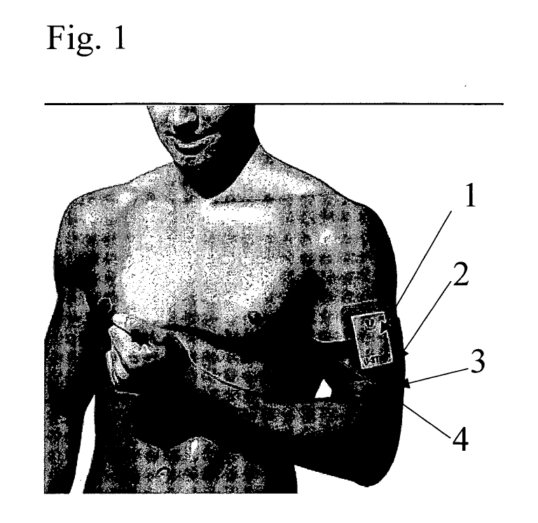

Referring now to FIG. 1, there is shown an illustration of an embodiment of the present invention as it is placed upon the arm of a patient. An enhanced TDD system may comprise a control device 1, which may be placed directly over a TDD 2. The control device 1, may include an ultrasound sensor. The control device 1 and patch 2 may be attached to the exterior of the patient's skin 3 by means of a strap 4, which may hold the control device 1 and TDD 2 in place. Power for the control device 1 may be provided by a power supply, which may be rechargeable, and may be located within the strap 4. Alternatively, a power supply may be contained within the control device 1 or provided by an external source.

The TDD system may be located on the arm of the patient, placed over the patient's chest, as in the case of nitroglycerin drug delivery, for example, or placed in any other more effective part of the patient's body as determined by the medical personnel administrating the TDD system.

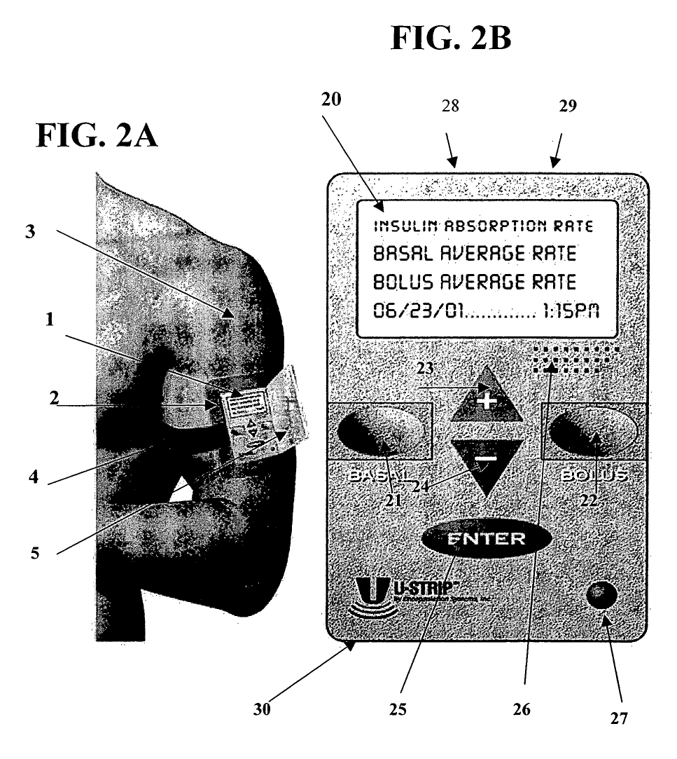

Referring now to FIG. 2A, there is shown an illustration of an embodiment of the present invention as it is placed upon the arm of a patient, wherein the cover 5 of the control device 1 is in an open position. Further, FIG. 2B illustrates an enlarged view of the control device 1, which may include, but is not limited to, a monitor, a sensor control, a power supply, modem, transducer 91, transducer array 90, and a processor. The control device 1 may further include: a display 20, a basal control button 21, a bolus control button 22, a scroll up button 23, a scroll down button 24, an entry key 25, an audible alarm 26, an alarm lamp 27, a modem port 28, a transducer port 29, and a test port 30.

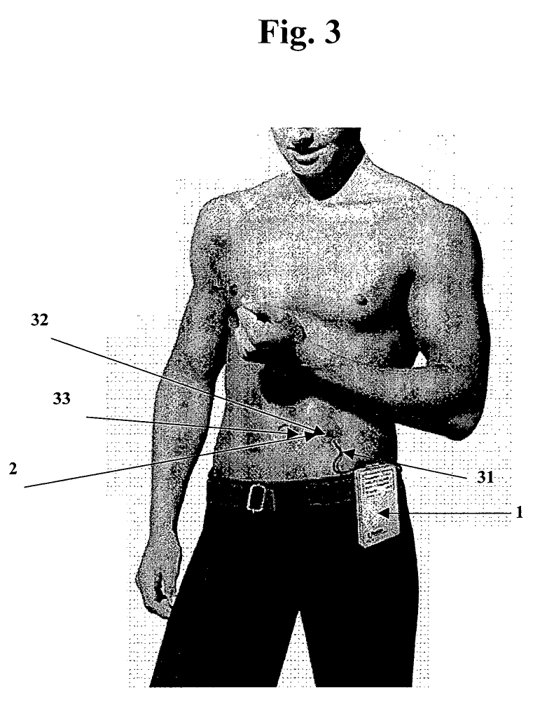

Referring now to FIG. 3, there is shown a further illustration of an embodiment of the present invention as it is placed upon the person of the patient remotely from the TDD 2 contact site. The TDD device may comprise a control device 1, which may be placed remotely away from the TDD device and communicatively coupled to the control device 1 by cable 31, for example. By way of non-limiting example only, a TDD device may be placed on the abdomen 33 and may be connected to the control device 1, by way of a cable 31, with the control device 1 attached to a patient's belt. The TDD 2 and control device 1 may, for example, be communicatively connected by way of an internal cable and/or wireless technology, for example.

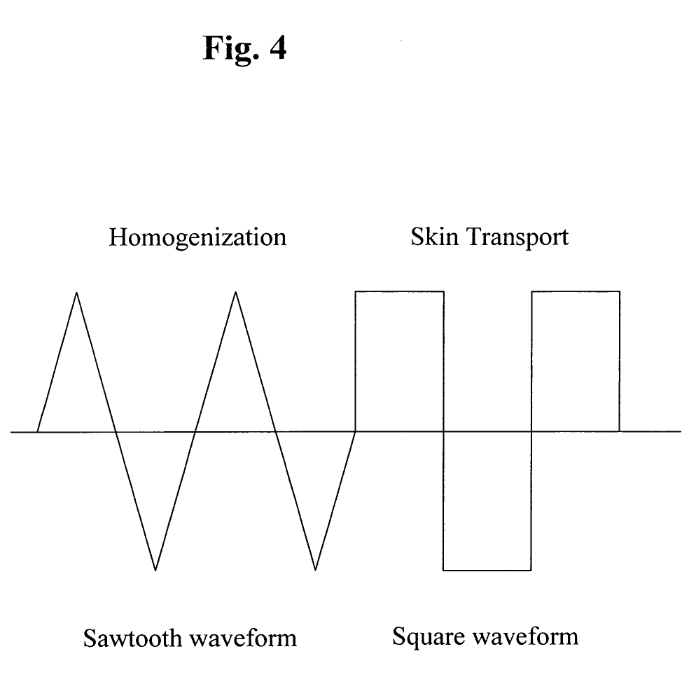

Referring now to FIG. 4, there is shown an alternating sonic waveform that may be produced by the present invention that may enhance the capability of the control device 1 of detecting the dose amount remaining within a TDD or delivered through the skin of a patient, wherein a combination of a sawtooth and a square waveform signal may be efficient at dose sensing when the echo return of a sonic transmission through a TDD is examined by the control device 1.

For example, the sawtooth waveform may include short periods indicating high energy coupled with short duration of pressure amplitude, which may lead to a vibration effect with the targeted pharmaceutical substance. This vibration effect may have a low heat potential and may have the effect of generating a precise energy signature upon reception of the echo signal for the ultrasonic sensor control within the control device 1. That energy signature may indicate the density of the patient's skin and the density of the drug contained within a TDD 2 utilizing an absorbent pad construction, for example.

When the sonic transmission converts to a square waveform, more energy may be releasing through the TDD 2 and through the skin of the patient, generating an echo signal with an energy signature, which may be equatable to a particular drug dose. The echo signal as converted by the control device 1, may indicate to the system how much liquid is remaining within the absorbent pad of a TDD. The degree of wetness may be equated to the number of milliliters of fluid originally within the pad and then the quantity remaining as time goes by. For example, a TDD may contain a cotton absorbent pad loaded with 1 ml of insulin solution, at a standard concentration of 100 units of insulin per ml, with each unit equaling 40 micrograms of active insulin. An echo sensor may indicate 100 units within a patch at 100% original liquid concentration. As time goes on, for example, it could indicate a 45% reduction in the original solution strengthen which would indicate that 45 units of insulin were deliverered from the patch and that 55 units remain.

In yet another embodiment, an echo signal may indicate 1.0 ml originally within the TDD's absorbent pad and 0% originally within the patient's upper layers of skin. As time goes by, the sensor control within the control device 1 may indicate dampness within the skin by measuring the density change of the fatty issue of the dermis layer within the skin. As the dermis layer becomes “wetter,” it may be an indication of drug flow from the TDD through the skin. The sensor may measure the electronic signature as the skin wettens via changes in density of the fatty tissue as it hydrates with the drug solution. By way of non-limiting example only, an initial density signature could indicate 0% drug flow. Upon calibration, the sensor control within the control device 1 may measure a 45% evacuation from the patch and a corresponding density signature change within the dermis layer, indicating 45 units of insulin had been delivered over a period of time.

Several combinations of sonic sensors are possible including: low frequency and low intensity ultrasound; and, high frequency, high intensity ultrasound; low frequency and high intensity ultrasound; high frequency, low intensity ultrasound, as long is care is taken that the sensor signal does not generate enhanced cavitation within the stratum corneum which could result in skin burning and damage to the drug either within the TDD or as it travels through the skin.

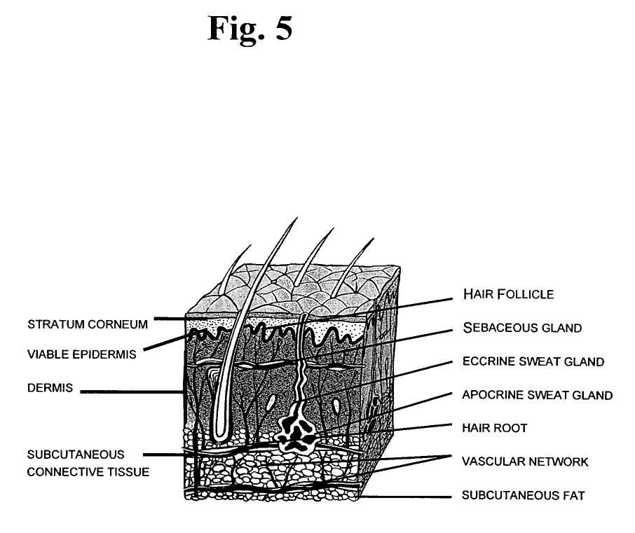

Referring now to FIG. 5, the present invention may direct a drug through the skin via the pathway afforded by sweat pores or a hair follicle. In traditional transdermal patches the drug is permeated through the skin via simple passive absorption. Further electronic means that can be used to “push” a drug through the skin include: ultrasonic, electrophoresis or iontophoresis techniques.

By way of non-limiting example only, the present invention may include an array which may include four transducers, with each transducer transmitting a driving force of 20-30 kHz ultrasonic frequency at 125 mW/sq. cm intensity using an alternating ultrasonic waveform consisting of 100 milliseconds on saw tooth waveform and then 100 milliseconds on square waveform before converting back to sawtooth. The sawtooth waveform component may enlarge the skin pores and the square waveform may drive the drug from the patch through the skin. Two of the transducers in the array may then convert to a higher frequency transmission about every 60 seconds. A frequency, 80 kHz, at the same intensity, 125 mW/sq. cm, sends an ultrasonic pulse through the absorbent pad of the patch which may last only 100 milliseconds using a sinusoidal waveform. The pulse may be similar to a sonar transmission and may have both a forward transmission and a return transmission or echo. The echo may then be received by the other two transducers in the array and may produce a voltage which may correspond to the degree of wetness of the liquid content on the absorbent pad. The same transducer array may be used to induce the movement of the drug from a TDD and deliver a drug or substance transdermally through the patient's skin, and to measure the dose delivered, using a driving setting of an alternating waveform and a about 20-30 kHz frequency. During the drug sensing mode, the frequency may convert to sine waves and jump to about 80 kHz. The echo voltage in the receiving transducers may measure the quantity of the liquid remaining within the absorbent pad portion of the patch.

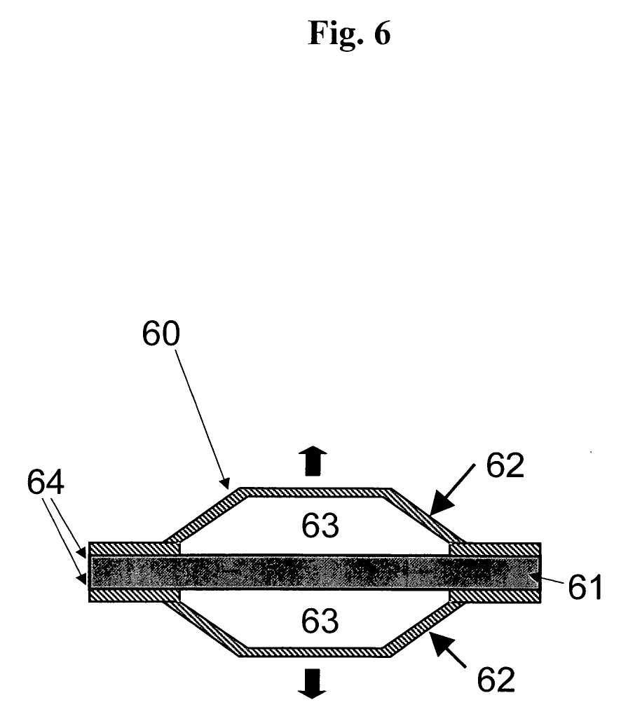

Referring now to FIG. 6, the present invention may include a cymbal type ultrasonic transducer 60. The cymbal transducer 60 may be based upon a piezoelectric disc 61 such as PZT4 (Piezokinetics Corp. Bellefonte, Pa.), connected to two metal caps 62 composed of titanium foil, for example. FIG. 6 further illustrates a hollow air space 63 between the piezoelectric disc 61 and the end caps 62. The end caps 62 may be connected to the piezoelectric disc 61 by a non-electrically conductive adhesive 64 and may form a bonded layered construction to the transducer 60. The cymbal transducer offers a thin, compact structure suited for portable ultrasonic drug delivery apparatus. Further, it offers greater efficiency for the conversion of electric power to acoustically radiated power.

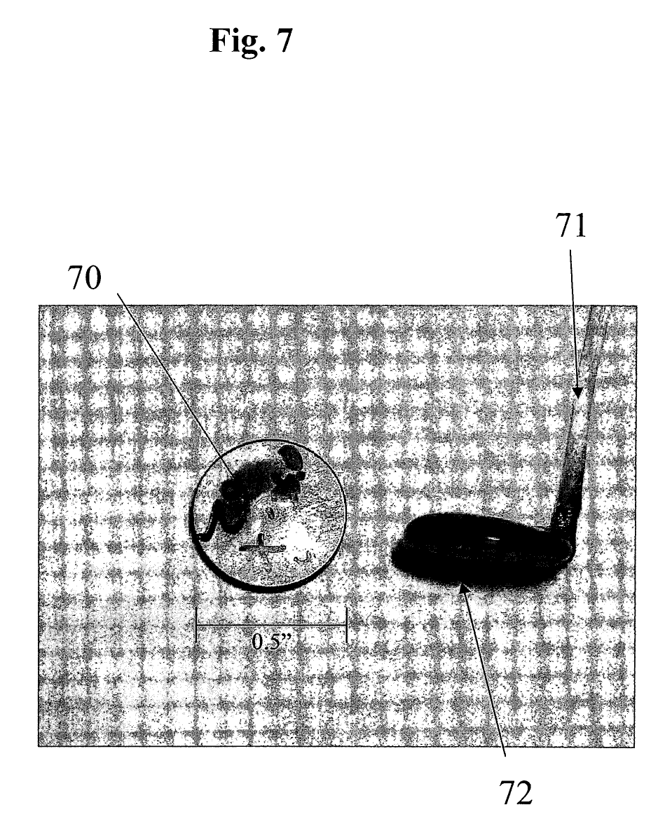

Referring now to FIG. 7, there is shown an embodiment of a cymbal transducer, which may be compact and small in size compared to the transducer element of the system. The size of the transducers may be about 0.5″ inches diameter. This size of transducer enables the transducers to fit within the dimensions of a TDD, for example. In addition, the small size of the transducer aids in the portability of the system. The transducer element 70 may be a cymbal type construction attached to a power cable 71. The transducer element 70 may be coated in a polymer housing 72, composed of uralite resin, for example, or any other material suitable for avoiding short circuits between the two metallic caps 62 and providing acoustic coupling for the sonic transmission.

The cymbal type transducer design may have a compact structure, with a small surface area, a high acoustic pressure and high acoustic intensity at low resonance frequency, and a high efficiency, making the system more energy efficient. The use of a low resonance frequency may avoid a high cavitation threshold, specifically, the intensity required to generate air bubbles within the stratum corneum of the patient's skin tissue. The cavitation threshold is directly proportional to the frequency applied so the choice of a low resonance frequency of the transducer permits a lower acoustical pressure applied to the surface of the skin and an echo pattern can be effected which will lead to a electronic character or signature, which in turn can be used to deduce changes in the density within the skin or the patch material, and thereby giving an indication of does original, dose remaining and a calculation of dose delivered.

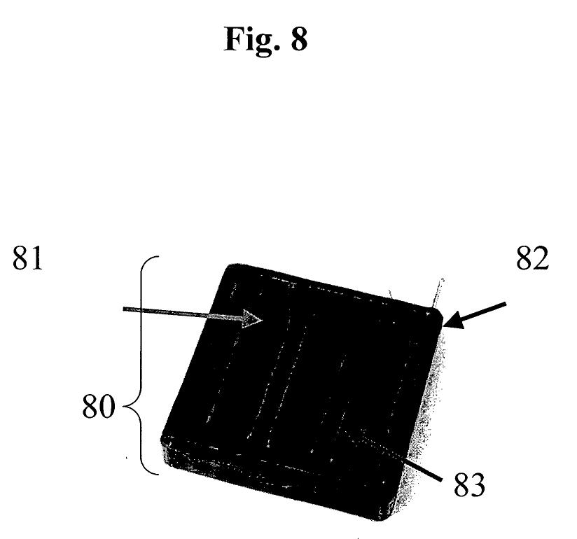

Referring now to FIG. 8, there is shown an array 80 consisting of at least one cymbal elements 81 arranged in a suitable pattern onto a substructure or encased within a polymer housing 82. The cymbal elements 81 are connected in parallel by a series of electrical connections 83. The array 80 may be then sealed in polymer potting material 82 composed of uralite, for example. The array may provide a portable, battery powered ultrasonic transmission, with suitable power to effect drug delivery via TDD.

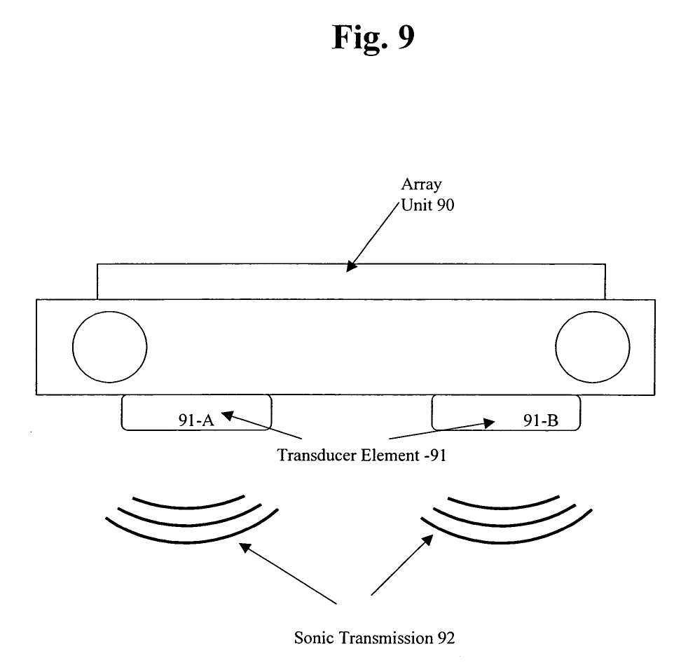

Referring now to FIG. 9, there is shown two miniature transducers 91A, 91B placed within or adjacent to a TDD 2 that may generate an ultrasonic transmission 92 capable of traveling through the TDD 2 or through the skin of the patient. The ultrasonic transmission 92 may generate an echo return pattern, which may be read and understood by an array unit 90 which may be connected to the TDD 2 or which may be separate from the TDD 2.

By way of non-limiting example only, a transdermal patch holder may be constructed according to the following dimensions: 2.73 in. Diameter X 0.63 in. height and may include an absorbent pad composed of cellulose material, such as, for example, model no. Vicell 9009, supplied by Buckeye Products Company, having a thickness of 0.92 mm/ply and a diameter of 1.6 in. The absorbent pad may then be loaded with a substance, for example, 0.75 ml of Humulin Reg. Insulin. The pad may then be sealed with saran film on both faces and placed into the a holder if applicable. A 4-element transducer array, model no. BKR-1007-37, for example, may be mated against the insulin loaded absorbent pad in a holder. The transducer array may have the ability to transmit two different ultrasonic transmissions: a driving force of 20-30 kHz ultrasonic frequency at 125 mW/sq. cm intensity using an alternating ultrasonic waveform consisting of 100 milliseconds on saw tooth waveform and then 100 milliseconds on square waveform before converting back to sawtooth (Setting A); and, a 80 kHz, at 125 mW/sq. cm, sinusoidal waveform for only 100 milliseconds, timed to send a pulse every 60 seconds through the absorbent pad (Setting B). Control of this function may be managed by an ultrasonic driver circuit Model No. ESI-25 B-1, provided by Encapsulation Systems Inc., and contained in the control device. Two of the transducers in the array are attached to an oscilloscope to measure peak-to-peak voltage of the measured echo pattern when the transducers are set at, for example, a 80 kHz, at 125 mW/sq. cm, sinusoidal waveform for only 100 milliseconds, timed to send a pulse every 60 seconds. The echo voltage in the sensor varied as the liquid loading, the insulin content, of the absorbent pad decreased.

By way of non-limiting example only, and as shown in Table 1, an absorbent pad was measured with an insulin load at a staring point and various weight loss measurements were made over time as the ultrasound at Setting A drove the insulin from the absorbent pad. These weight measurements were compared to the peak-to-peak voltage over the echo transducers to develop a voltage match to the liquid content remaining in the pad at that time. In this example, the starting weight of the pad un-loaded was 0.0995 grams with a loaded weight (0.75 ml of insulin, equivalent to 75 units of insulin) of 0.8114 grams and a starting voltage of a pulse through the fully loaded pad of 222 millivolts.

TABLE 1

| | | | | | | U/S | | |
|---|---|---|---|---|---|---|---|---|
| | Absorbent Pad | | | | | Activation | | |
| | Tare wt. In | Liquid loading | Absorbent Pad | Liquid Loss | Percentage | Time in | Echo Volts P-P | Change in Echo Voltage |
| Run | grams | wt. In grams | Weight | in grams | of load | Minutes | in mV | across transducers |
| 1 | 0.0995 | 0.7119 | 0.8114 | 0 | 100% | 0 | 222 | 0.00% |
| 2 | 0.0995 | 0.5519 | 0.6514 | 0.16 | 22% | 5 | 201 | 90.54% |
| 3 | 0.0995 | 0.4719 | 0.5714 | 0.24 | 34% | 10 | 175 | 78.83% |
| 4 | 0.0995 | 0.3919 | 0.4914 | 0.32 | 45% | 15 | 156 | 70.27% |
| 5 | 0.0995 | 0.2819 | 0.3814 | 0.43 | 60% | 20 | 132 | 59.46% |

As illustrated by Table 1, for example, at 5 minutes of continuous ultrasound treatment 22% of the starting weight of the insulin had been lost from the pad and the corresponding echo voltage of the sensor had dropped from 222 mV to 201 mV, a decrease of nearly 10%. By minute 20 of continuous exposure the liquid loss was 43 ml comparable to an Echo voltage of 132 mV. Thus, a sensor may trigger off of the liquid content within the patch and the liquid content loss over time can be calculated by a microprocessor in the control device to indicate the dose delivered from the patch in relation to the ultrasonic voltage data coming from the sensor arrangement.

The sensor may determine the dose remaining in the patch at any given time, and therefore may calculate the dose delivered from the patch. It can be inferred that the dose delivered went into the patient to provide a dose controlling mechanism for ultrasonic drug delivery.

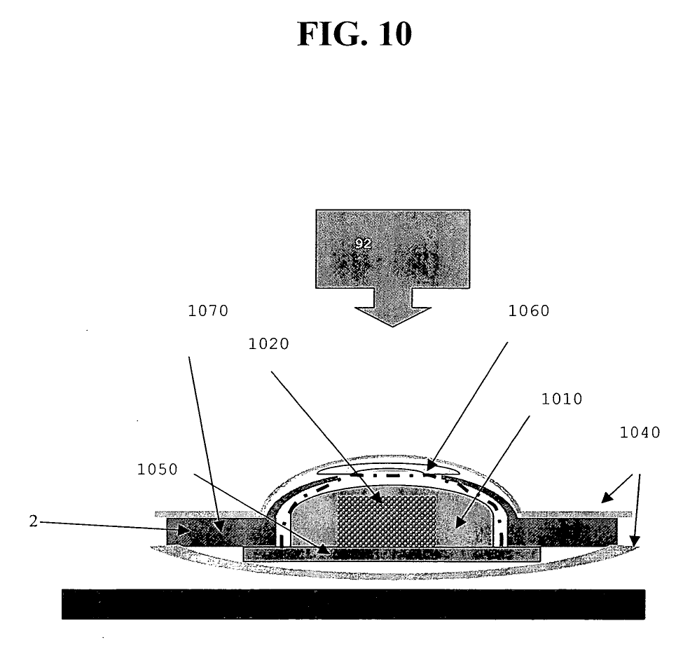

Referring now to FIG. 10, there is shown an embodiment of a TDD 2, which may allow for the penetration of an ultra sonic transmission 92. Absorbent pad 1010 is used to store the drug 1020. An ultrasonic transmission 92 may generate a dose metering effect within the absorbent pad 1010 by generating an echo pattern within the absorbent pad 1010. The electric intensity of the echo pattern may be used to determine the degree of dampness or the moisture content of the absorbent pad 1010. Knowing the drug 1020 concentration within a liquid suspension may allow for the quantity of the drug 1020 to be determined. Comparing a start position of known dose and electric matching signal of the ultrasonic signal, the dose delivered from the absorbent pad 1010 over time may be determined. Additionally, an ultrasonic transmission 92 may be intensified and sent through the patient's skin, which may provide a reading of the dose actually delivered past the stratum corneum skin barrier layer. The TDD 2 may also include a protective covering of peel-away film 1040, a semi-permeable film 1050, which may allow for the release of medicant due to the introduction of an ultrasonic transmission 92, for example, a sonic membrane 1060, and a suitable material to act as a platform 1070 for the TDD 2.

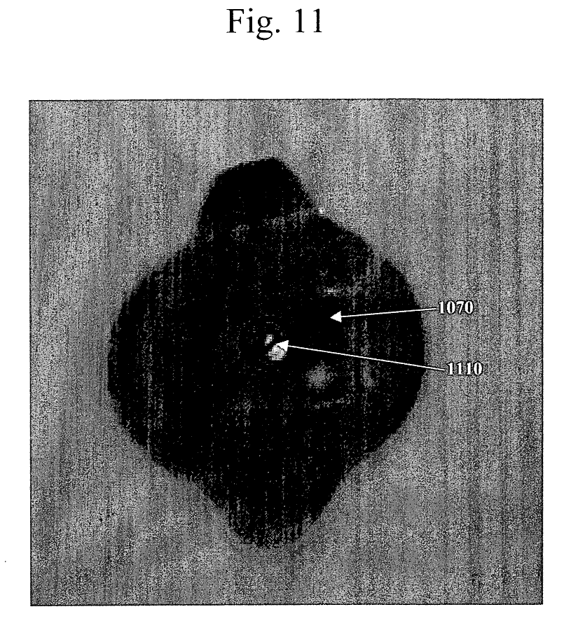

Referring now to FIG. 11, there is shown a transducer 1110 or an array of transducers that may be built directly within the TDD 2 and imbedded into the platform 1070.

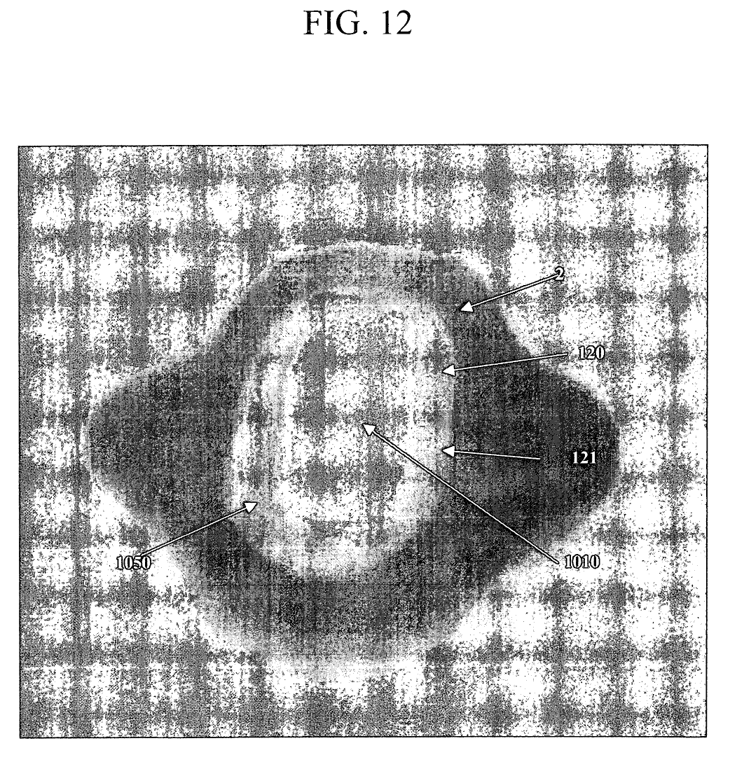

Referring now to FIG. 12, there is shown an embodiment of a TDD 2 further illustrating a semi-permeable film 1050 at least partially covering the absorbent pad 1010 and well 120 and secured to the TDD 2 by sealant 121.

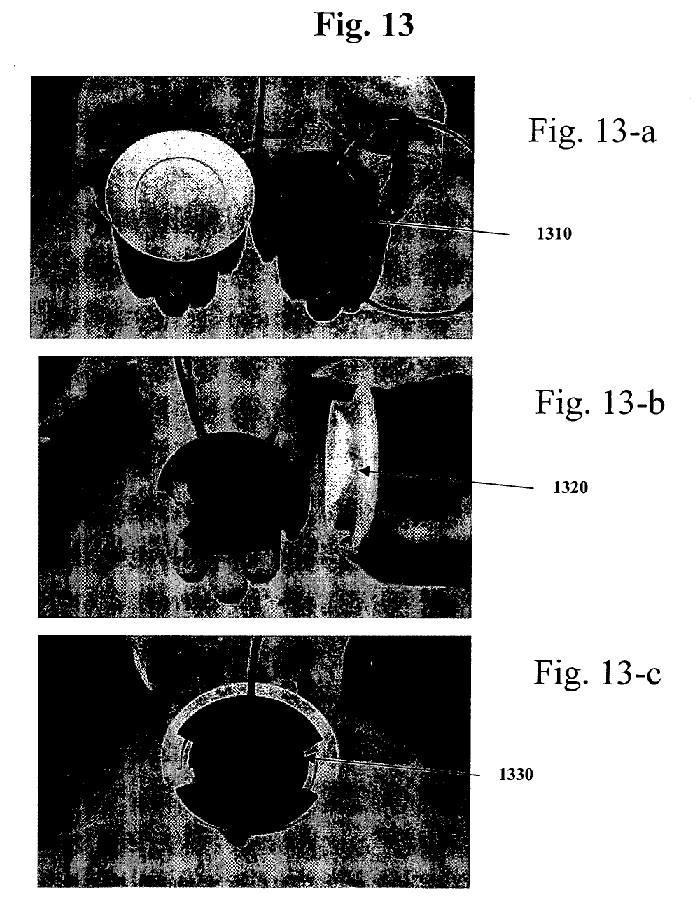

Referring now to FIGS. 13A. 13B and 13C, there is shown a two-part TDD 2, which may consist of a transducer array contained within a transducer coupler 1310, which connects to a patch cap 1320. The transducer coupler 1310 sends an ultrasonic transmission 92 through the absorbent pad 1010 in the patch cap 1320 and through the patient's skin in much the same manner as it would through a film 1050 based TDD 2, for the purposes of measuring: the original dose within the TDD 2; the dose liberated form the TDD 2; the dose remaining within the TDD 2 compared to the starting quantity; the dose at a particular time actually delivered to the patient, through the skin; and, the cumulative dose actually delivered to the patient.

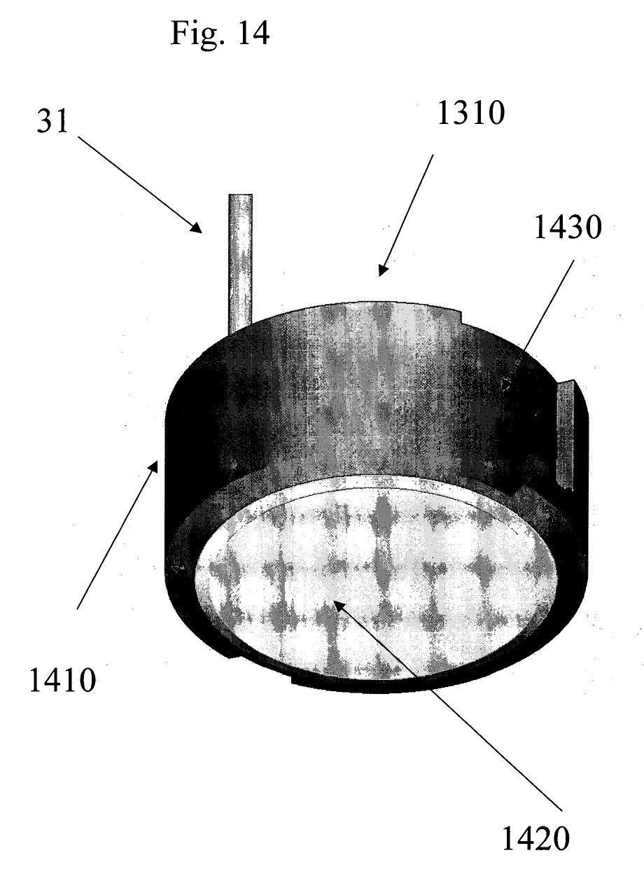

Referring now to FIG. 14, there is shown an embodiment of a transducer coupler 1310, which may include a cable 31, a housing 1410, a sonic face plate 1420, and a cap connect groove 1430.

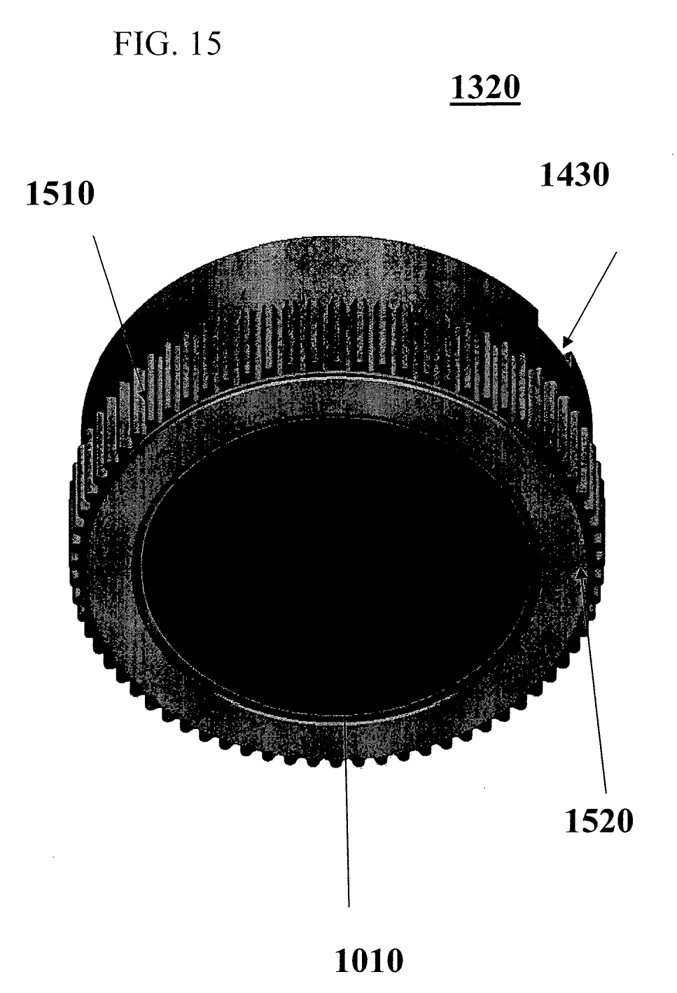

Referring now to FIG. 15, there is shown a bottom view of the patch cap 1320, which may include a threaded ring 1510, and adhesive ring 1520, a cap connect groove 1430, and an absorbent pad 1010, wherein the absorbent pad 1010 may be communicatively coupled to the skin.

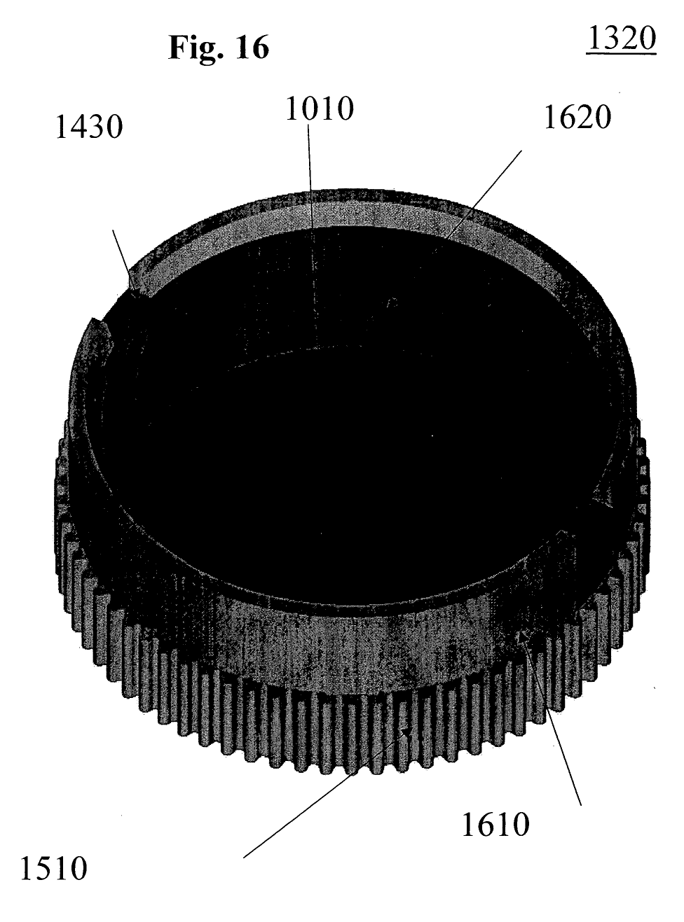

Referring now to FIG. 16, there is shown a top view of the patch cap 1320, which may include a cap connector groove 1430, a threaded ring 1510, an outer snap ring 1610, and inner snap ring 1620, and an absorbent pad 1010.

This invention describes the use of acoustic energy in the ultrasonic range, which can be generated by a vibrating plate or by an ultrasonic transducer.

Ultrasound is defined as a sound of frequency of between 20 kHz and 10 MHz, with intensities of between 0 and 3 W/cm2.

The use of low frequency ultrasound, ideally from 20-100 kHz, using alternating waveform (from sawtooth to square wave), cymbal type transducers which enable a battery power ultrasonic transmission, through a transdermal patch or TDD, generating an echo pattern which when returned to the transducer, can be used to deduce the dose remaining within the patch material or to deduce the dose which has been actually delivered through the skin of the patient who is wearing a TDD.

The present invention may, by way of non-limiting example only, may be capable of delivering large molecule compounds, such as, for example, compounds above 4,000 Daltons in molecular weight, across the skin barrier layer such that the present invention may provide for the measured administration of insulin doses delivered through an electronic transdermal delivery device, such as an ultrasonic delivery system.

While an ultrasonic sensor is described, it should be noted that other electronic means may be applied to measure the dose within a patch or TDD and the dose delivered to the patient via a transdermal delivery device or modified form of transdermal patch.

The disclosure herein is directed to the variations and modifications of the elements and methods of the invention disclosed that will be apparent to those skilled in the art in light of the disclosure herein. Thus, it is intended that the present invention covers the modifications and variations of this invention, provided those modifications and variations come within the scope of the appended claims and the equivalents thereof.

1. A transdermal substance delivery device, comprising:
at least one ultrasonic transducer for generating at least one ultrasonic transmission for inducing movement of at least one substance into a tissue;
said at least one ultrasonic transmission and at least one sensor positioned with said at least one transducer to sense reflected ultrasonic transmissions; and
wherein, said sensed ultrasonic transmissions are indicative of substance actually moved into said tissue.

2. The transdermal substance delivery device of claim 1, wherein said ultrasound has a frequency in the range of about 20 KHz to 30 MHz.

3. The transdermal substance delivery device of claim 1, wherein said ultrasonic transmission has an intensity of about 125-mW/sq. cm to 3.0 W/sq. cm.

4. The transdermal substance delivery device of claim 1, wherein said ultrasound utilizes an alternating waveform.

5. The transdermal substance delivery device of claim 4, wherein said alternating waveform comprises a sawtooth waveform.

6. The transdermal substance delivery device of claim 4, wherein said alternating waveform comprises a square waveform.

7. The transdermal substance delivery device of claim 1, wherein said ultrasound is applied substantially continuously.

8. The transdermal substance delivery device of claim 1, wherein said ultrasound is pulsed.

9. The transdermal substance delivery device of claim 1, further comprising a control device.

10. A method for transdermal substance delivery, comprising:
generating at least one ultrasonic transmission from at least one ultrasonic transducer for inducing movement of at least one substance into a tissue;
positioning said at least one ultrasonic transmission and at least one sensor positioned with said at least one transducer to sense reflected ultrasonic transmissions; and
wherein, said sensed ultrasonic transmissions are indicative of substance actually moved into said tissue.

11. The method of claim 10, wherein said ultrasonic transmission has a frequency in the range of about 20 KHz to 30 MHz.

12. The method of claim 10, wherein said ultrasonic transmission has an intensity of about 125-mW/sq. cm to 3.0 W/sq. cm.

13. The method of claim 10, wherein said ultrasonic transmission utilizes an alternating waveform.

14. The method of claim 13, wherein said alternating waveform comprises a sawtooth waveform.

15. The method of claim 13, wherein said alternating waveform comprises a square waveform.

16. The method of claim 10, wherein said ultrasonic transmission is applied substantially continuously.

17. The method of claim 10, wherein said ultrasonic transmission is pulsed.

18. The method of claim 10, further comprising a control device.
